(19) United States
(12) Patent Application Publication
(10) Pub. No.: US 2020/0268172 A1
(43) Pub. Date: Aug. 27, 2020

(54) ANTI-SIDS INFANT CRIB

(71) Applicant: MADOC EL MALI, RISHON LEZION (IL)

(72) Inventor: MADOC EL MALI, RISHON LEZION (IL)

(21) Appl. No.: 16/871,073

(22) Filed: May 11, 2020

Related U.S. Application Data
(63) Continuation-in-part of application No. 16/172,901, filed on Oct. 29, 2018.

(30) Foreign Application Priority Data
Oct. 30, 2017 (IL) 255340

(51) Int. Cl. A47D 15/00; A61B 5/113; A61B 5/00; A47D 9/00; A61B 5/01; A47J 36/24

(52) U.S. Cl. CPC A47D 15/008; A47D 15/001; A61B 5/113; A61B 2505/07; A47D 9/00; A61B 5/01; A47J 36/2433; A61B 5/4818

(57) ABSTRACT

An anti-SIDS infant crib, including a crib, a limited-stretchability belt to be worn by an infant lying in the crib, a processor, one or more sensors, in communication with the processor, where the sensors being disposed on the belt, thereby being in contact with the infant for allowing metering SIDS-related sensing of the infant, a pair of limited-stretchability straps each connected to an opposite side of the belt, for allowing the infant to turn on its side while preventing from the infant to turn upside-down, a vibrator disposed in the belt, the vibrator being operable by the processor upon detecting a deep-sleep situation, thereby stopping the deep-sleep situation of the infant, and a remote control in wireless communication with the processor, the remote control being adapted to alert a user upon exceeding a predetermined threshold of the sensors.

### TECHNICAL FIELD

The invention relates to the field of SIDS prevention.

### BACKGROUND

“Sudden infant death syndrome (SIDS), also known as cot death or crib death, is the sudden unexplained death of a child less than one year of age. SIDS usually occurs during sleep. Placing an infant to sleep while lying on the stomach or the side increases the risk. This increased risk is greatest at two to three months of age.” (From Wikipedia)

U.S. Pat. No. 8,333,560 discloses a portable fan that can attach to a baby crib in order to provide air movement across an infant in order to prevent the likelihood of sudden infant death syndrome.

U.S. Pat. No. 5,389,037 discloses a method and apparatus for reducing respiratory abnormalities in infants and the incidence of crib death by providing a flow of room air to a sleeping infant's environment to stimulate breathing. In an embodiment of the invention, room air is delivered into a crib through an air plenum that is removably attached to the vertical bars of a crib and positioned within 1 cm to 20 cm of the infant's mouth, nose, larynx and trachea. The flow of room air from the air plenum safely assists the respiration of an infant with inadequate shallow end tidal volume, decreases rebreathing of expired carbon dioxide from the nose, mouth, oral cavity and trachea, prevents pockets of increasing carbon dioxide from developing within the cushions and beddings surrounding the infant, and decreases the likelihood of overheating and other conditions associated with sudden infant death syndrome, apnea syndromes and hypoventilation.

U.S. Pat. No. 5,483,711 discloses a safety pad or mattress such as for use in a crib prevents sudden infant death syndrome by ensuring an oxygenated breathing space beneath the infant. Reticulated foam or other air permeable material is made into the shape of a pad or even a mattress and covered with a fitted open weave fabric covering. An air tube is embedded in the pad or mattress and interconnected with an air pump which circulates fresh, i.e., oxygenated, air in a breathing space formed beneath an infant by the air permeable mattress. The forced air circulation flushes any exhaled carbon dioxide from the breathing space, even when the infant is face down or otherwise in a prone position on the mattress, to prevent carbon dioxide poisoning.

All the methods described above have not yet provided satisfactory solutions to the problem of preventing crib death.

### SUMMARY

In one embodiment of the invention an anti-SIDS infant crib is provided, including a crib (10), a limited-stretchability belt (30), to be worn by an infant (40) lying in the crib, a processor (20), one or more sensors (38), in communication with the processor (20), where the sensors being disposed on the belt, thereby being in contact with the infant for allowing metering SIDS-related sensing of the infant, a pair of limited-stretchability straps (34) each connected to an opposite side of the belt, for allowing the infant to turn on its side while preventing from the infant to turn upside-down, a vibrator disposed in the belt, the vibrator being operable by the processor upon detecting a deep-sleep situation, thereby stopping the deep-sleep situation of the infant, and a remote control (50) in wireless communication (60) with the processor (20), the remote control being adapted to alert a user upon exceeding a predetermined threshold of the sensors, thereby providing a system for diminishing SIDS probability and alerting a user when a SIDS situation is detected, while leaving a pelvis of the infant free of movement limitations.

In one embodiment of the invention the sensors (38) include a camera (16) and microphone (62), for taking images of an infant lying in the crib, and displaying thereof on a remote display.

The crib may further include a multimedia system (42), disposed at the crib's side, for displaying content to the infant.

The crib according may further include a screen (12), for displaying to the infant video presentations.

The crib may further include an adjustable mattress (14), for allowing adapting thereof so that an infant lying in the crib can view the screen.

The crib may further include a baby bottle heater facility (22) controllable by the processor (20), for heating a baby bottle (36) disposed therein.

According to one embodiment of the invention, the one or more sensors (38) that come in contact with an infant body or leotard include a heartbeat rate sensor.

According to one embodiment of the invention, the one or more sensors (38) that come in contact with an infant body or leotard include a breath rate sensor.

According to one embodiment of the invention, the one or more sensors (38) that come in contact with an infant body or leotard include an oxygen amount sensor in blood.

According to one embodiment of the invention, the one or more sensors (38) that come in contact with an infant body or leotard include a hygrometer, for detecting if a diaper of the infant needs to be changed.

According to one embodiment of the invention, the one or more sensors (38) that come in contact with an infant body or leotard include a thermometer, for metering a temperature of the infant.

According to one embodiment of the invention, the one or more sensors (38) include a thermometer, for measuring a surrounding temperature.

According to one embodiment of the invention, the one or more sensors (38) include a microphone, and the processor is further adapted to detect an infant's cry.

According to one embodiment of the invention, the one or more sensors (38) include a length meter in a form of a track (28) disposed at the inner side of the crib's walls, and a length counter (26) slideable along the track, thereby allowing measuring a height of the infant when lying in the crib.

According to one embodiment of the invention, the one or more sensors (38) include a weight meter, for metering a weight of the infant.

The crib may further include memory for storing results of measurements, thereby allowing diagnosing abnormal development of an infant lying in the crib.

The crib may further include a transmitter for transmitting measurements' results to a remote database, for deducing combinations and thresholds in which an alert has to be issued, and updating the processor of thresholds and combinations of alerting states.

According to one embodiment of the invention the limited-stretchability belt (30) further includes a display for displaying SIDS measurements of sensors of the belt, states and instructions to a user thereof.

According to one embodiment of the invention the limited-stretchability belt (30) is adjustable.

According to one embodiment of the invention, the limited-stretchability straps (34) are adjustable.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

### BRIEF DESCRIPTION OF THE DRAWINGS

Embodiments, features, and aspects of the invention are described herein in conjunction with the following drawings in which:

FIG. 1 is a perspective view which pictorially illustrates an anti-SIDS infant crib, according to one embodiment of the invention.

FIG. 2 is a front view thereof;

FIG. 3a is a longitudinal sectional view of an anti-SIDS infant crib, according to one embodiment of the invention;

FIG. 3b is a longitudinal sectional view of an anti-SIDS infant crib, such that the left side of the mattress (in the illustration's orientation) is inclined, according to one embodiment of the invention;

FIG. 4 pictorially illustrates an infant lying in an anti-SIDS infant crib, according to one embodiment of the invention;

FIG. 5 pictorially illustrates a remote control of an anti-SIDS infant crib, according to one embodiment of the invention;

FIG. 6 is a block diagram illustrating the data flow in an anti-SIDS infant crib, according to one embodiment of the invention;

FIG. 7a is a latitudinal cross-section of an anti-SIDS infant crib in which the infant lies on his back, according to one embodiment of the invention;

FIG. 7b is a latitudinal cross-section of an anti-SIDS infant crib in which the infant tries to turn upside-down while the straps 34 prevents it, according to one embodiment of the invention;

Each of FIGS. 8a and 8b schematically illustrates an operation of the limited-stretchability, according to one embodiment of the invention; and

FIG. 9 schematically illustrates an inner side of an anti-SIDS infant belt 30, according to one embodiment of the invention.

It should be understood that the drawings are not necessarily drawn to scale.

### DETAILED DESCRIPTION

The invention will be understood from the following detailed description of embodiments which are meant to be descriptive and not limiting. For the sake of brevity, some well-known features, methods, systems, procedures, components, circuits, and so on, are not described in detail.

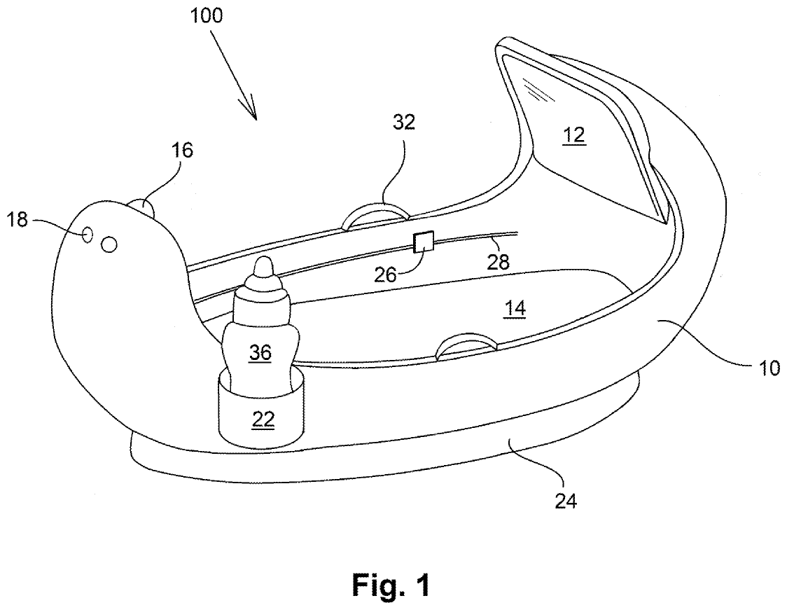

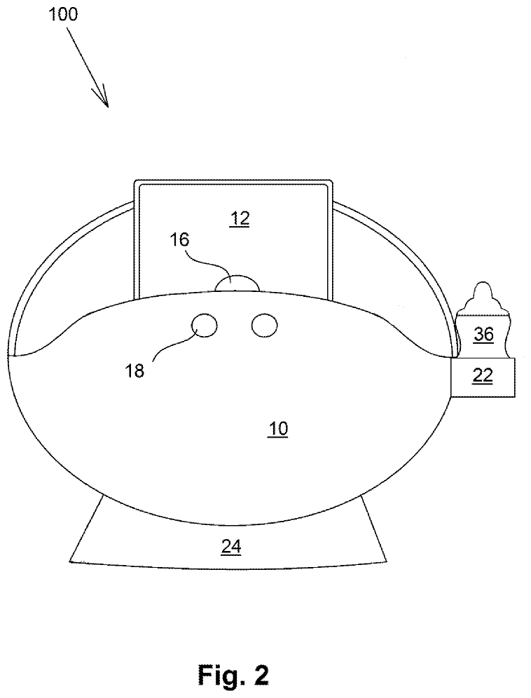

FIG. 1 is a perspective view which pictorially illustrates an anti-SIDS infant crib, according to one embodiment of the invention. FIG. 2 is a front view thereof.

Preferably, the contour of the crib 10 is of an egg, which people, especially adults, associate with primacy.

The crib comprises carrying handles 32, by which a parent can move the crib and place it in another location.

A display is disposed against the face of the infant lying in the crib, for displaying thereon graphical presentations, films, and the like, which may interest the infant. The display may be a computer monitor, a tablet computer, a television display, and the like.

The crib comprises a mattress 14 which is composed of two or more parts, so that the angle between them can be changed. In this way, the position of the infant lying on the mattress can be adjusted to allow him a better view of the screen 12.

The angle may be changed manually or by electromechanical means (not illustrated).

The angle change may be activated remotely or by local input means such as by button, knobs, and so on (not illustrated).

Preferably, in the crib is installed a multimedia system comprising at least audio display and speakers, which preferably are disposed at the sides of the infant's head. The multimedia system may use a display, such as screen 12.

Above the head of the infant is disposed a camera 16, through which a parent can watch the infant at a distance. Preferably, the camera comprises night vision capability, in order to allow the parent to clearly see the infant at night time.

A baby bottle heater facility 22 is installed on a wall of the crib, whether inside or outside the crib. A parent can put the baby food bottle inside the facility, and at feeding time the bottle will be warm. Preferably, the bottle heater comprises input means (knob, slide, etc.) for adjusting the required heating temperature.

The crib comprises a length meter, for measuring the infant's length when lying (i.e., its height). As will be detailed hereinafter, the length meter may be embodied as a length counter 26 (trailer) slideable along a track 28 at the inner side of the crib's walls. The length counter (trailer) may be as a longitudinal digital micrometer. Thus, the parent places the trailer near the infant top, sets the counter to zero, and drags the trailer to the bottom of the infant. While dragging the trailer, the distance is displayed on its digital display. In order to allow a parent to point on the top and bottom points of the infant, the counter 26 may further comprise a vertical bar (not illustrated).

A user may press a “SAVE” button in order to store the recent metered measurement and its date in a database.

At the outer side of the crib is installed one or more lamps 18 for providing background light around the crib. The color and intensity of the lighting of the lamps may be controlled by appropriate input means (knobs and the like).

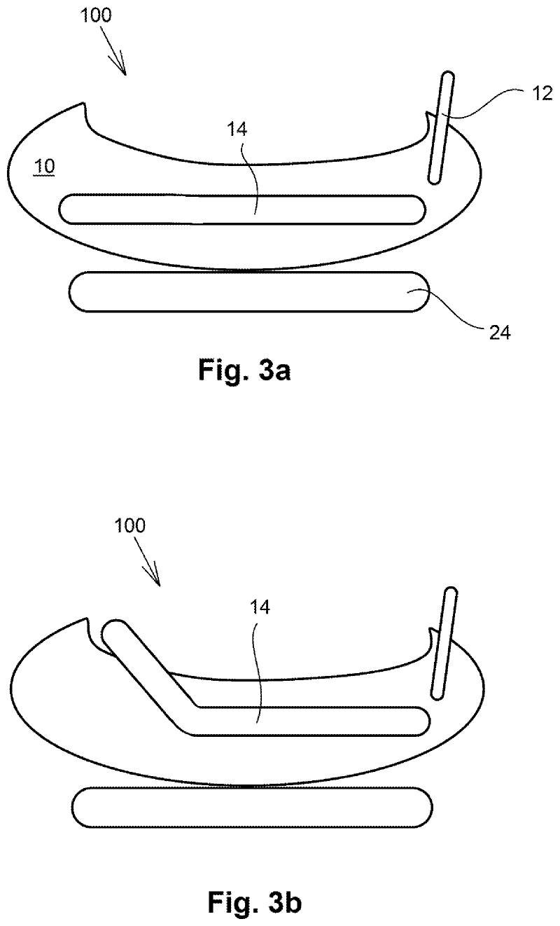

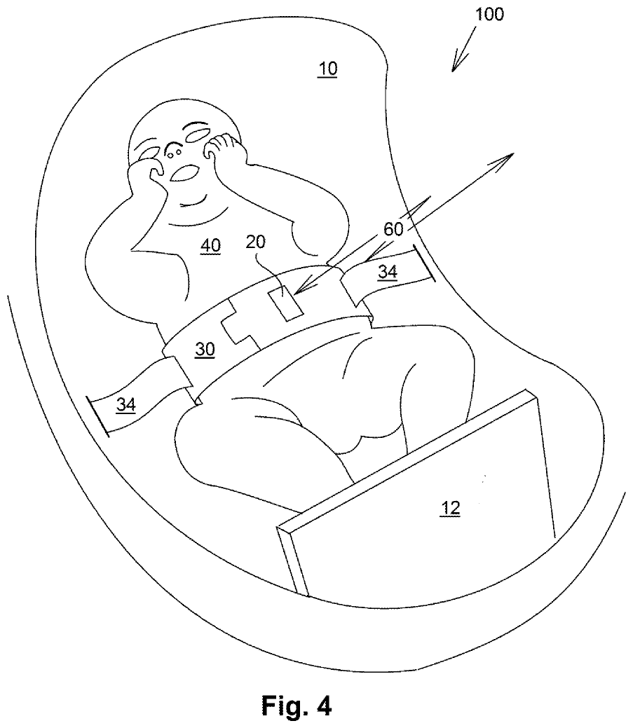

FIG. 3a is a longitudinal sectional view of an anti-SIDS infant crib, according to one embodiment of the invention.

As mentioned, the crib comprises a mattress 14 which is composed of two or more parts, so that the angle between them can be changed. In this way, the position of the infant lying on the mattress can be adjusted to allow him a better view of the screen 12.

FIG. 3b is a longitudinal sectional view of an anti-SIDS infant crib, such that the left side of the mattress (in the illustration's orientation) is inclined, according to one embodiment of the invention.

The inclination angle may be changed manually or by electromechanical means (not illustrated).

The inclination angle may be controlled remotely or by local input means (such as by button, knobs, and so on (not illustrated).

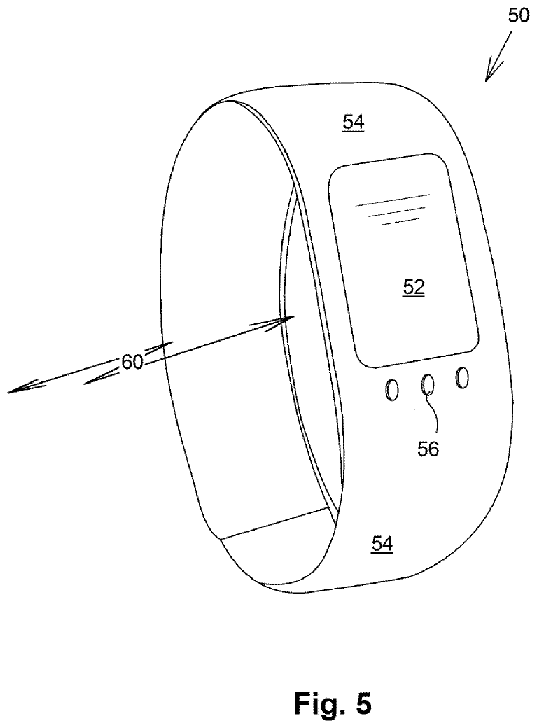

FIG. 4 pictorially illustrates an infant lying in an anti-SIDS infant crib, according to one embodiment of the invention.

The infant 40 wears a belt 30 on which may be installed a plurality of sensors (not shown), such as heartbeat rate sensor, breath rate sensor, hygrometer, thermometer, microphone, a weight meter, and the like.

Disposing the sensors on the belt, which embraces the infant, allow a direct contact with the infant's organs, and therefore provide a more reliable detection of SIDS situations or situations that may result with SIDS.

The sensors are connected to a processor 20 which communicates with a remote control (not shown) via wireless communication 60, with a smart phone and the like (not shown). Preferably, the remote control is also used as an alerting device. Alternatively, the alerting device may be embedded in the processor.

When one of the sensors senses an exceptional measurement (i.e., the metered value is compared to a predefined threshold, a combination of a plurality of sensors' readings indicate alerting situations, and so on), the system alerts the parent via the alerting device.

The heartbeat sensor and breath rate sensor may be embedded in the belt. The hygrometer may be disposed in the diaper of the infant, and connected to the processor 20 by wired or unwired communication means. The weight sensor may be disposed under the mattress, or on the mattress. The same regards the microphone. The sound pattern sampled by the microphone may be compared to a pattern of baby cries, and when the infant cries, the parent may be alerted. The alert may include an indication of the intensity of the cry. For example, the density beep sounds may indicate the strength of the cry, the intensity of the alert may indicate the strength of the cry, and so on.

One of the major features of the invention is the ability to prevent an infant lying in the crib from rolling on his side or stomach. Such means may be limited-stretchability straps 34 for connecting the belt 30 to the walls of the crib, a Velcro fastener (not shown) and so on. As mentioned, keeping the infant lying on his back decreases the chance of the infant dying from crib death.

The term “deep-sleep” refers herein to a sleep situation characterized by, for example, snoring and breathing difficulty, pauses in breathing, restlessness, coughing or choking, sweating profusely, etc.

These parameters can be measured by sensors, which can be disposed in the belt. For example, the audio signal produced by the breath of an infant can be sampled by a microphone (e.g., inhaling and exhaling rhythm), analyzed by a processor, which can detect breath disorders, etc., for instance by comparing the sampled results to expected ones.

FIG. 5 pictorially illustrates a remote control of an anti-SIDS infant crib, according to one embodiment of the invention.

The remote control 50 comprises wrist belt 54, a display 52, and operational buttons 56. The wrist belt 54 allows a user to wear the remote control 50 on his wrist.

The remote control 50 also comprises a processor (not shown) which communicates with the processor 20 (not shown) on the crib via wireless communication 60.

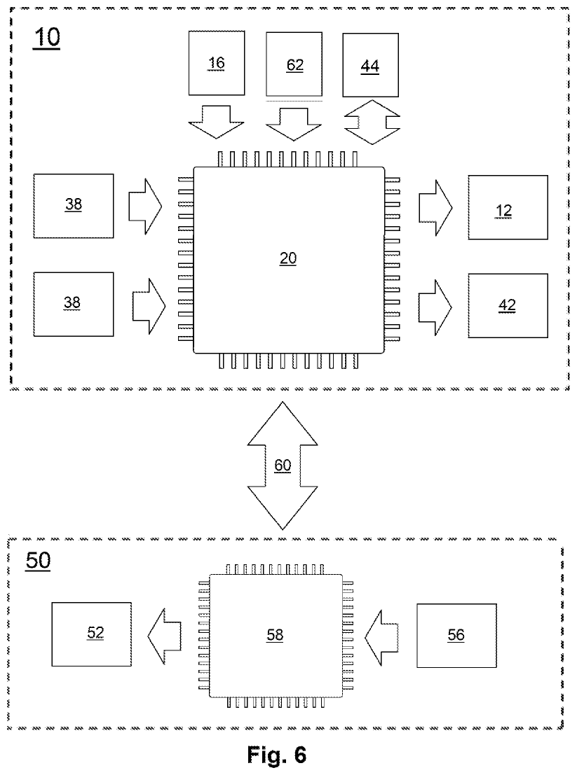

FIG. 6 is a block diagram illustrating the data flow in an anti-SIDS infant crib, according to one embodiment of the invention.

At the crib site is installed a processor 20 which gets input from sensors 38, camera 16, microphone 62, and the like.

The sensors 38 may be a heartbeat rate sensor, breath rate sensor, hygrometer, microphone, a weight sensor, and the like. The processor compares the metered values from the sensors, and compares each value to a predefined threshold. When a metered value passes beyond the threshold, an alert signal is transmitted to remote control 50 via wireless communication channel 60.

As per sensors such as heartbeat rate sensor, breath rate sensor, hygrometer, weight sensor, and the like, which meters a parameter such as heartbeat rate, the metered parameter is compared to a predefined threshold thereof, and if the metered value exceeds the threshold, an alert is sent to the remote control 50.

A memory 44 may store the metered values. The stored values may be used for determining regular behavior, so that un-regular values may indicate a SIDS situation. Of course, the stored values may be used for research, transmitted to a remote database, etc.

As per the microphone, the processor may use a DSP (Digital Signal Processing) processor (not shown) for detecting from the sampled audio signal if the infant is crying, the cry intensity, and the like.

According to another embodiment of the invention, the strength of the sampled sound is metered. If the cry's strength passes beyond a predefined threshold, then an alert signal is transmitted to the remote control 50.

In addition, the processor may transmit to the remote control a reminder for feeding the infant, changing the infant's diaper, and the like.

The multimedia system 42 comprises at least audio display circuitry for playing audio signals, and speakers, which preferably are disposed at the sides of the infant's head. The multimedia system may also use a display, such as screen 12, thereby allowing displaying movies. The multimedia system may use external memory in which are stored multimedia files, thereby allowing a parent to change the displayed content.

It should be noted that the processor does not necessarily have to be installed on the infant's belt, but in any place at the crib's side.

The remote control 50 comprises a processor 58, which gets input from operational buttons and displays output signals by output device(s) 52, such as a display and speaker. The operational buttons are used for setting the thresholds, reminders, and the like.

According to one embodiment of the invention, the remote control 50 is embodied by a smart phone running a corresponding software application. This spares the development time from manufacturers, using an additional device, and so on.

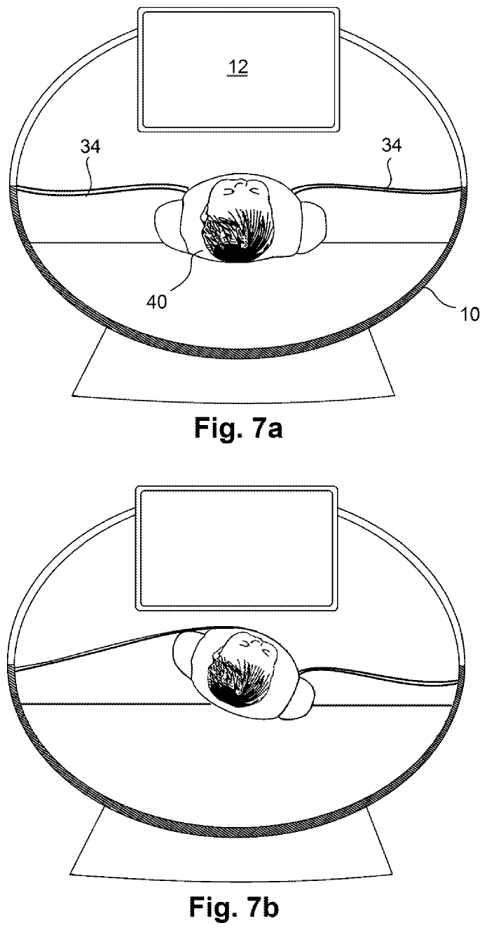

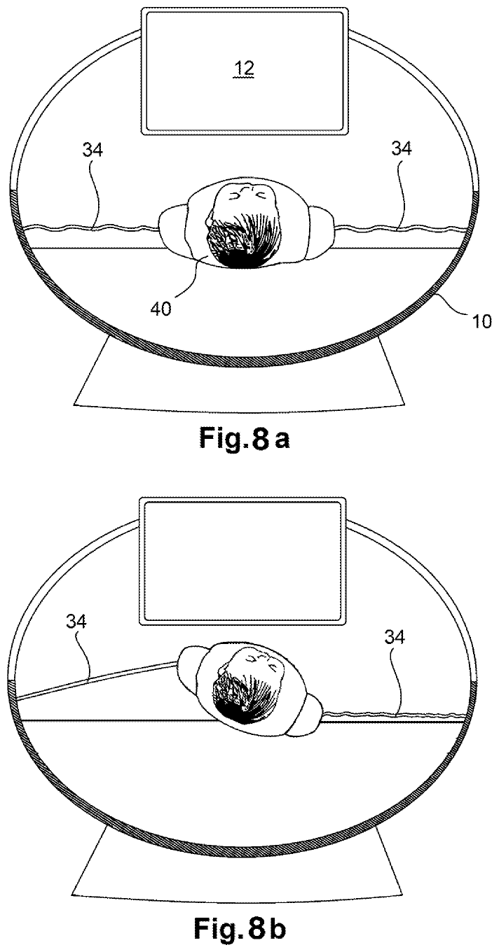

FIG. 7a is a latitudinal cross-section of an anti-SIDS infant crib in which the infant lies on his back, according to one embodiment of the invention.

FIG. 7b is a latitudinal cross-section of an anti-SIDS infant crib in which the infant tries to turn upside-down while the straps 34 prevents it, according to one embodiment of the invention.

As mentioned above, one of the major features of the invention is the ability to prevent an infant lying in the crib from rolling on his side or stomach. Such means may be limited-stretchability straps 34 for connecting the belt 30 to the walls of the crib, a Velcro fastener 64, and so on. As mentioned, keeping the infant lying on his back decreases the chance of the infant dying from SIDS.

As illustrated in FIG. 7b, the left strap 34 (in the figure's orientation) is tensed while the right belt is loose, thereby preventing the infant from turning upside-down on his stomach. On the one hand, the infant can change its position, but on the other hand he cannot turn upside-down.

Alternatively, the bottom side of the belt can comprise a Velcro fastener, while the mattress may comprise the corresponding Velcro patch. The drawback of this solution is that the rolling ability of the infant is limited.

The term “limited-stretchability” refers herein to the ability of an object to be stretched to a limited extension, and return to its non-stretched form after being released. Lycra fabric is an example of a fabric having a limited-stretchability. On one hand it can be stretched, and when being released it returns to its original form.

According to one embodiment of the invention, the belt 34 is made of a limited-stretchability material. Thus, when using this kind of material for a belt and straps that connect the infant to a crib, the infant has a limited ability to turn around. As such, the limited-stretchability material can be selected such that the infant can roll on its side without much effort, but cannot turn over.

Each of FIGS. 8a and 8b schematically illustrates a stretching stage of a strap 34, according to one embodiment of the invention.

According to this embodiment of the invention, the infant strap 34 is made of limited-stretchability material, such as Lycra fabric.

As illustrated, when the infant 40 turns on its right, the left strap 34 is stretched to its limit, and therefore prevents from the infant to turn beyond this limit, i.e., prevents from the infant to turn upside-down.

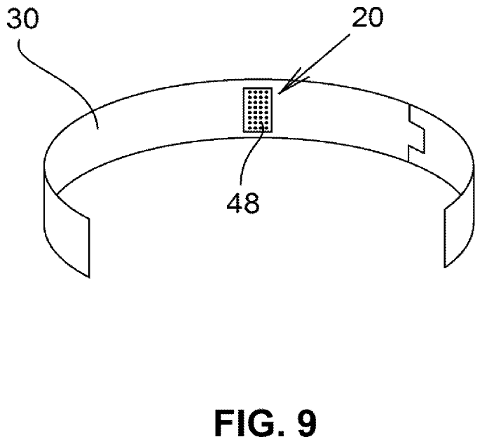

FIG. 9 schematically illustrates an inner side of an anti-SIDS infant belt 30, according to one embodiment of the invention.

As illustrated, the processor 20 comprises sensors 38 disposed at the inner side of the belt 30, such that when an infant wears the belt, the sensors touch his body or leotard, and therefore can sense the temperature of the infant, if he sweats, his heartbeats rate, etc.

For the sake of brevity, the illustration is schematic, and therefore is not detailed.

In the figures and/or description herein, the following reference numerals (Reference Signs List) have been mentioned:
numeral 10 denotes a crib;
numeral 12 denotes a screen;
numeral 14 denotes a mattress;
numeral 16 denotes a camera;
numeral 18 denotes a background light lamp;
numeral 20 denotes a processor, i.e., a circuitry that responds to and processes basic computing/electronic instructions;
numeral 22 denotes a baby bottle heater facility;
numeral 24 denotes a base of crib 10;
numeral 26 denotes a length counter;
numeral 28 denotes a track;
numeral 30 denotes a limited-stretchability belt;
numeral 32 denotes a handle;
numeral 34 denotes limited-stretchability straps;
numeral 36 denotes a baby bottle;
numeral 38 denotes a sensor;
numeral 40 denotes an infant;
numeral 42 denotes multimedia system;
numeral 44 denotes a memory (in a form of a chip, hard driver, etc.);
numeral 50 denotes a remote control;
numeral 52 denotes output device(s) of remote control 50, such as screen and speakers;
numeral 54 denotes a wrist belt of remote control 50;
numeral 56 denotes operational buttons;
numeral 58 denotes a processor;
numeral 60 denotes wireless communication;
numeral 62 denotes a microphone; and
numeral 64 denotes a Velcro fastener.

In the description herein, the following references have been mentioned: U.S. Pat. Nos. 8,333,560, 5,389,037, and 5,483,711.

The foregoing description and illustrations of the embodiments of the invention has been presented for the purposes of illustration. It is not intended to be exhaustive or to limit the invention to the above description in any form.

Any term that has been defined above and used in the claims, should be interpreted according to this definition.

The reference numbers in the claims are not a part of the claims, but rather used for facilitating the reading thereof. These reference numbers should not be interpreted as limiting the claims in any form.

What is claimed is:

1. An anti-SIDS infant crib, comprising:
a crib (10);
a limited-stretchability belt (30), to be worn by an infant (40) lying in said crib;
a processor (20);
one or more sensors (38), in communication with said processor (20), wherein said sensors being disposed on said belt, thereby being in contact with said infant for allowing metering SIDS-related sensing of said infant;
a pair of limited-stretchability straps (34) each connected to an opposite side of said belt, for allowing said infant to turn on its side while preventing from said infant to turn upside-down;
a vibrator disposed in said belt, said vibrator being operable by said processor upon detecting a deep-sleep situation, thereby stopping said deep-sleep situation of said infant; and
a remote control (50) in wireless communication (60) with said processor (20), said remote control being adapted to alert a user upon exceeding a predetermined threshold of said sensors,
thereby providing a system for diminishing SIDS probability and alerting a user when a SIDS situation is detected, while leaving a pelvis of said infant free of movement limitations.

2. An anti-SIDS infant crib according to claim 1, wherein said sensors (38) include a camera (16) and microphone (62), for taking images of an infant lying in said crib, and displaying thereof on a remote display.

3. An anti-SIDS infant crib according to claim 1, further comprising a multimedia system (42), disposed at said crib's side, for displaying content to said infant.

4. An anti-SIDS infant crib according to claim 3, further comprising a screen (12), for displaying to said infant video presentations.

5. An anti-SIDS infant crib according to claim 4, further comprising an adjustable mattress (14), for allowing adapting thereof so that an infant lying in said crib can view said screen.

6. An anti-SIDS infant crib according to claim 4, further comprising a baby bottle heater facility (22) controllable by said processor (20), for heating a baby bottle (36) disposed therein.

7. An anti-SIDS infant crib according to claim 1, wherein said one or more sensors (38) that come in contact with an infant body or leotard include a heartbeat rate sensor.

8. An anti-SIDS infant crib according to claim 1, wherein said one or more sensors (38) that come in contact with an infant body or leotard include a breath rate sensor.

9. An anti-SIDS infant crib according to claim 1, wherein said one or more sensors (38) that come in contact with an infant body or leotard include an oxygen amount sensor in blood.

10. An anti-SIDS infant crib according to claim 1, wherein said one or more sensors (38) that come in contact with an infant body or leotard include a hygrometer, for detecting if a diaper of said infant needs to be changed.

11. An anti-SIDS infant crib according to claim 1, wherein said one or more sensors (38) that come in contact with an infant body or leotard include a thermometer, for metering a temperature of said infant.

12. An anti-SIDS infant crib according to claim 1, wherein said one or more sensors (38) include a thermometer, for measuring a surrounding temperature.

13. An anti-SIDS infant crib according to claim 1, wherein said one or more sensors (38) include a microphone, and said processor is further adapted to detect an infant's cry.

14. An anti-SIDS infant crib according to claim 1, wherein said one or more sensors (38) include a length meter in a form of a track (28) disposed at the inner side of the crib's walls, and a length counter (26) slideable along said track, thereby allowing measuring a height of said infant when lying in said crib.

15. An anti-SIDS infant crib according to claim 1, wherein said one or more sensors (38) include a weight meter, for metering a weight of said infant.

16. An anti-SIDS infant crib according to claim 1, further comprising memory for storing results of measurements, thereby allowing diagnosing abnormal development of an infant lying in said crib.

17. An anti-SIDS infant crib according to claim 1, further comprising a transmitter for transmitting measurements' results to a remote database, for deducing combinations and thresholds in which an alert has to be issued, and updating said processor of thresholds and combinations of alerting states.

18. An anti-SIDS infant crib according to claim 1, wherein said limited-stretchability belt (30) further comprising a display, for displaying SIDS measurements of sensors of said belt, states and instructions to a user thereof.

19. An anti-SIDS infant crib according to claim 1, wherein said limited-stretchability belt (30) is adjustable.

20. An anti-SIDS infant crib according to claim 1, wherein said limited-stretchability straps (34) are adjustable.
